(19) United States
(12) Patent Application Publication
(10) Pub. No.: US 2025/0046439 A1
(43) Pub. Date: Feb. 6, 2025

(54) SYSTEMS AND METHODS FOR PATIENT HEALTHCARE UTILITY

(71) Applicant: DOCSNAP, INC., Atlanta, GA (US)

(72) Inventor: Anthony J. Mari, Marietta, GA (US)

(73) Assignee: DOCSNAP, INC., Atlanta, GA (US)

(21) Appl. No.: 18/794,666

(22) Filed: Aug. 5, 2024

Related U.S. Application Data
(60) Provisional application No. 63/530,754, filed on Aug. 4, 2023.

(51) Int. Cl. G16H 40/20; G06F 16/25; G16H 10/60; G16H 80/00

(52) U.S. Cl. CPC G16H 40/20; G06F 16/258; G16H 10/60; G16H 80/00

(57) ABSTRACT

A computer-implemented method including receiving a first request for electronic health records (EHRs). The method also can include sending a second request to a first health information exchange to cause the first health information exchange to send one or more third requests for at least some of the EHRs from second health information exchanges through a health information network. The method additionally can include receiving the EHRs from the first health information exchange. The method further can include causing the EHRs to be processed into a requested format. The method additionally can include providing the EHRs, in the requested format, to a patient portal application for display to a user. Other embodiments are described.

### CROSS-REFERENCE TO RELATED APPLICATION

This application claims the benefit of U.S. Provisional Application No. 63/530,754, filed Aug. 4, 2023. U.S. Provisional Application No. 63/530,754 is incorporated herein by reference in its entirety.

### TECHNICAL FIELD

This disclosure relates generally to systems and methods for a patient healthcare utility.

### BACKGROUND

Electronic health records (EHRs) have become an part of modern healthcare systems, offering numerous benefits such as improved patient care, enhanced communication between healthcare providers, and increased efficiency in medical practices. However, the widespread adoption of EHRs has also introduced challenges related to data interoperability, patient access, and information exchange between different healthcare organizations. Traditionally, patient medical records were maintained in paper form and stored at individual healthcare facilities. This approach often resulted in fragmented and incomplete patient histories, as information was not easily shared between different providers or institutions. The transition to electronic records has aimed to address these issues, but the healthcare industry still faces obstacles in achieving seamless data exchange and comprehensive patient records. One significant challenge is the existence of multiple EHR systems and data formats used by various healthcare providers and organizations. This diversity can lead to difficulties in sharing and interpreting patient information across different platforms. Additionally, patients often receive care from multiple providers over time, resulting in their health information being dispersed across various systems and locations. Furthermore, patients have historically had limited access to their own health records, often facing bureaucratic hurdles and delays when attempting to obtain their medical information. This lack of easy access can impede patients' ability to obtain their healthcare records and take an active role in managing their health and sharing relevant information with their healthcare providers.

### BRIEF DESCRIPTION OF THE DRAWINGS

To facilitate further description of the embodiments, the following drawings are provided in which:

FIG. 1 illustrates a front elevational view of a computer system that is suitable for implementing an embodiment of the system disclosed in FIG. 3;

FIG. 2 illustrates a representative block diagram of an example of the elements included in the circuit boards inside a chassis of the computer system of FIG. 1;

FIG. 3 illustrates a block diagram of a system that can be employed for providing a patient healthcare utility, and a data flow of messages between the components of the system, according to an embodiment;

FIG. 4 illustrates a block diagram of a system that can be employed for providing a patient healthcare utility, and a data flow of activities involving the components of the system, according to an embodiment;

FIG. 5 illustrates a flow chart of a publish-subscribe messaging model using a simple notification service, according to an embodiment;

FIG. 6 illustrates display screens for a mobile device for a patient, including a login screen, a notification screen, a user account screen, and a messages screen;

FIG. 7 illustrates display screens for a mobile device for a patient, including a Consolidated CDA (Clinical Document Architecture) screen, a conditions listing page, a condition status page, and a condition details page;

FIG. 8 illustrates display screens for a mobile device for a patient, including a provider screen, a family members screen, a home screen, and a messages screen;

FIG. 9 illustrates display screens for a mobile device for a patient, including a provider screen, a provider details screen, a visit options screen, and a virtual visit screen;

FIG. 10 illustrates display screens for a mobile device for a patient, including a Consolidated CDA screen, an action screen, and a message screen;

FIG. 11 illustrates display screens for a mobile device for a patient, including a home screen, a dashboard screen, and an insurance options screen;

FIG. 12 illustrates display screens for a mobile device for a patient, including an insurance options screen, a coverage screen, and a medical coverage screen;

FIG. 13 illustrates display screens for a mobile device for a patient, including a messages screen, a menu screen, a claims screen, and a claim details screen;

FIG. 14 illustrates a CDA in human-readable format, prescription options, administration options, and plans/materials;

FIG. 15 illustrates display screens for a mobile device for a provider, including a login screen, a messages screen, and a sent records screen;

FIG. 16 illustrates display screens for a mobile device for a provider, including message listing screens, a message screen, and a send message screen;

FIG. 17 illustrates a webpage display screen that shows a summary view of patient longitudinal medical records;

FIG. 18 illustrates a webpage display screen that shows a timeline view for a patient's medical history;

FIG. 19 illustrates a webpage display screen that shows medications for a patient; and

FIG. 20 illustrates a flow chart for a method of retrieving and providing EHRs through a patient healthcare utility, according to an embodiment.

For simplicity and clarity of illustration, the drawing figures herein illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the invention described herein are, for example, capable of operation in other orientations than those illustrated or otherwise described herein.

The terms “couple,” “coupled,” “couples,” “coupling,” and the like should be broadly understood and refer to connecting two or more elements or signals, electrically, mechanically or otherwise. Two or more electrical elements may be electrically coupled, but not mechanically or otherwise coupled; two or more mechanical elements may be mechanically coupled, but not electrically or otherwise coupled; two or more electrical elements may be mechanically coupled, but not electrically or otherwise coupled. Coupling (whether mechanical, electrical, or otherwise) may be for any length of time, e.g., permanent or semi-permanent or only for an instant.

“Electrical coupling” and the like should be broadly understood and include coupling involving any electrical signal, whether a power signal, a data signal, and/or other types or combinations of electrical signals. “Mechanical coupling” and the like should be broadly understood and include mechanical coupling of all types. The absence of the word “removably,” “removable,” and the like near the word “coupled,” and the like does not mean that the coupling, etc. in question is or is not removable.

### DESCRIPTION OF EXAMPLES OF EMBODIMENTS

Various embodiments include a computer-implemented method. The method can include receiving a first request for electronic health records (EHRs). The method also can include sending a second request to a first health information exchange to cause the first health information exchange to send one or more third requests for at least some of the EHRs from second health information exchanges through a health information network. The method additionally can include receiving the EHRs from the first health information exchange. The method further can include causing the EHRs to be processed into a requested format. The method additionally can include providing the EHRs, in the requested format, to a patient portal application for display to a user.

A number of embodiments include a system including one or more processors and one or more non-transitory computer-readable media storing computing instructions that, when executed on the one or more processors, cause the one or more processors to perform certain operations. The operations can include receiving a first request for electronic health records (EHRs). The operations also can include sending a second request to a first health information exchange to cause the first health information exchange to send one or more third requests for at least some of the EHRs from second health information exchanges through a health information network. The operations additionally can include receiving the EHRs from the first health information exchange. The operations further can include causing the EHRs to be processed into a requested format. The operations additionally can include providing the EHRs, in the requested format, to a patient portal application for display to a user.

A number of embodiments include one or more non-transitory computer-readable media storing computing instructions that, when executed on one or more processors, cause the one or more processors to perform certain operations. The operations can include receiving a first request for electronic health records (EHRs). The operations also can include sending a second request to a first health information exchange to cause the first health information exchange to send one or more third requests for at least some of the EHRs from second health information exchanges through a health information network. The operations additionally can include receiving the EHRs from the first health information exchange. The operations further can include causing the EHRs to be processed into a requested format. The operations additionally can include providing the EHRs, in the requested format, to a patient portal application for display to a user.

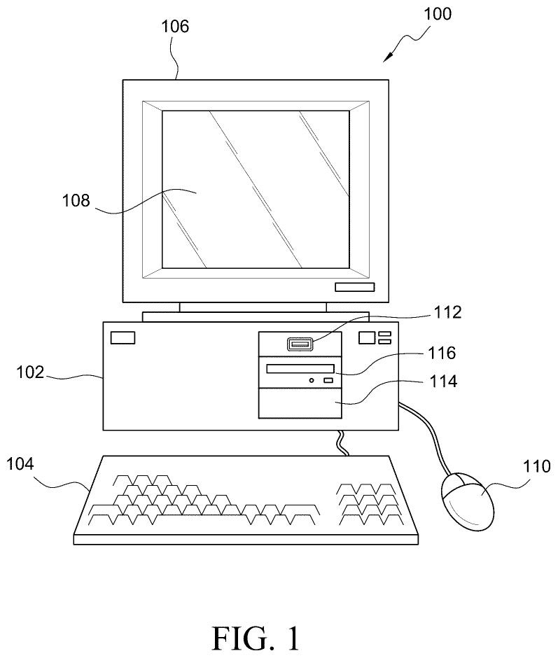

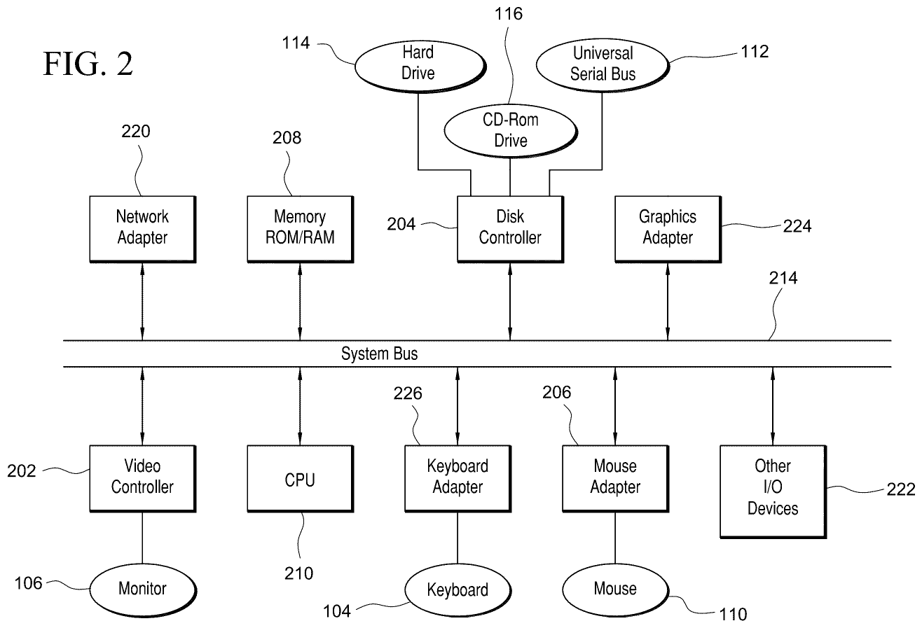

Turning to the drawings, FIG. 1 illustrates an exemplary embodiment of a computer system 100, all of which or a portion of which can be suitable for (i) implementing part or all of one or more embodiments of the techniques, methods, and systems and/or (ii) implementing and/or operating part or all of one or more embodiments of the non-transitory computer readable media described herein. As an example, a different or separate one of computer system 100 (and its internal components, or one or more elements of computer system 100) can be suitable for implementing part or all of the techniques described herein. Computer system 100 can comprise chassis 102 containing one or more circuit boards (not shown), a Universal Serial Bus (USB) port 112, a Compact Disc Read-Only Memory (CD-ROM) and/or Digital Video Disc (DVD) drive 116, and a hard drive 114. A representative block diagram of the elements included on the circuit boards inside chassis 102 is shown in FIG. 2. A central processing unit (CPU) 210 in FIG. 2 is coupled to a system bus 214 in FIG. 2. In various embodiments, the architecture of CPU 210 can be compliant with any of a variety of commercially distributed architecture families.

Continuing with FIG. 2, system bus 214 also is coupled to memory storage unit 208 that includes both read only memory (ROM) and random access memory (RAM). Non-volatile portions of memory storage unit 208 or the ROM can be encoded with a boot code sequence suitable for restoring computer system 100 (FIG. 1) to a functional state after a system reset. In addition, memory storage unit 208 can include microcode such as a Basic Input-Output System (BIOS). In some examples, the one or more memory storage units of the various embodiments disclosed herein can include memory storage unit 208, a USB-equipped electronic device (e.g., an external memory storage unit (not shown) coupled to universal serial bus (USB) port 112 (FIGS. 1-2)), hard drive 114 (FIGS. 1-2), and/or CD-ROM, DVD, Blu-Ray, or other suitable media, such as media configured to be used in CD-ROM and/or DVD drive 116 (FIGS. 1-2). Non-volatile or non-transitory memory storage unit(s) refer to the portions of the memory storage units(s) that are non-volatile memory and not a transitory signal. In the same or different examples, the one or more memory storage units of the various embodiments disclosed herein can include an operating system, which can be a software program that manages the hardware and software resources of a computer and/or a computer network. The operating system can perform basic tasks such as, for example, controlling and allocating memory, prioritizing the processing of instructions, controlling input and output devices, facilitating networking, and managing files. Exemplary operating systems can include one or more of the following: (i) Microsoft® Windows® operating system (OS) by Microsoft Corp. of Redmond, Washington, United States of America, (ii) Mac® OS X by Apple Inc. of Cupertino, California, United States of America, (iii) UNIX® OS, and (iv) Linux® OS. Further exemplary operating systems can comprise one of the following: (i) the iOS® operating system by Apple Inc. of Cupertino, California, United States of America, (ii) the Blackberry® operating system by Research In Motion (RIM) of Waterloo, Ontario, Canada, (iii) the WebOS operating system by LG Electronics of Seoul, South Korea, (iv) the Android™ operating system developed by Google, of Mountain View, California, United States of America, (v) the Windows Mobile™ operating system by Microsoft Corp. of Redmond, Washington, United States of America, or (vi) the Symbian™ operating system by Accenture PLC of Dublin, Ireland.

As used herein, “processor” and/or “processing module” means any type of computational circuit, such as but not limited to a microprocessor, a microcontroller, a controller, a complex instruction set computing (CISC) microprocessor, a reduced instruction set computing (RISC) microprocessor, a very long instruction word (VLIW) microprocessor, a graphics processor, a digital signal processor, or any other type of processor or processing circuit capable of performing the desired functions. In some examples, the one or more processors of the various embodiments disclosed herein can comprise CPU 210.

In the depicted embodiment of FIG. 2, various I/O devices such as a disk controller 204, a graphics adapter 224, a video controller 202, a keyboard adapter 226, a mouse adapter 206, a network adapter 220, and other I/O devices 222 can be coupled to system bus 214. Keyboard adapter 226 and mouse adapter 206 are coupled to a keyboard 104 (FIGS. 1-2) and a mouse 110 (FIGS. 1-2), respectively, of computer system 100 (FIG. 1). While graphics adapter 224 and video controller 202 are indicated as distinct units in FIG. 2, video controller 202 can be integrated into graphics adapter 224, or vice versa in other embodiments. Video controller 202 is suitable for refreshing a monitor 106 (FIGS. 1-2) to display images on a screen 108 (FIG. 1) of computer system 100 (FIG. 1). Disk controller 204 can control hard drive 114 (FIGS. 1-2), USB port 112 (FIGS. 1-2), and CD-ROM and/or DVD drive 116 (FIGS. 1-2). In other embodiments, distinct units can be used to control each of these devices separately.

In some embodiments, network adapter 220 can comprise and/or be implemented as a WNIC (wireless network interface controller) card (not shown) plugged or coupled to an expansion port (not shown) in computer system 100 (FIG. 1). In other embodiments, the WNIC card can be a wireless network card built into computer system 100 (FIG. 1). A wireless network adapter can be built into computer system 100 (FIG. 1) by having wireless communication capabilities integrated into the motherboard chipset (not shown), or implemented via one or more dedicated wireless communication chips (not shown), connected through a PCI (peripheral component interconnector) or a PCI express bus of computer system 100 (FIG. 1) or USB port 112 (FIGS. 1-2). In other embodiments, network adapter 220 can comprise and/or be implemented as a wired network interface controller card (not shown).

Although many other components of computer system 100 (FIG. 1) are not shown, such components and their interconnection are well known to those of ordinary skill in the art. Accordingly, further details concerning the construction and composition of computer system 100 (FIG. 1) and the circuit boards inside chassis 102 (FIG. 1) are not discussed herein.

When computer system 100 in FIG. 1 is running, program instructions stored on a USB drive in USB port 112, on a CD-ROM or DVD in CD-ROM and/or DVD drive 116, on hard drive 114, or in memory storage unit 208 (FIG. 2) are executed by CPU 210 (FIG. 2). A portion of the program instructions, stored on these devices, can be suitable for carrying out all or at least part of the techniques described herein. In various embodiments, computer system 100 can be reprogrammed with one or more modules, system, applications, and/or databases, such as those described herein, to convert a general purpose computer to a special purpose computer. For purposes of illustration, programs and other executable program components are shown herein as discrete systems, although it is understood that such programs and components may reside at various times in different storage components of computing system 100, and can be executed by CPU 210. Alternatively, or in addition to, the systems and procedures described herein can be implemented in hardware, or a combination of hardware, software, and/or firmware. For example, one or more application specific integrated circuits (ASICs) or Field Programmable Gate Arrays (FPGAs) can be programmed to carry out one or more of the systems and procedures described herein. For example, one or more of the programs and/or executable program components described herein can be implemented in one or more ASICs or FPGAs.

Although computer system 100 is illustrated as a desktop computer in FIG. 1, there can be examples where computer system 100 may take a different form factor while still having functional elements similar to those described for computer system 100. In some embodiments, computer system 100 may comprise a single computer, a single server, or a cluster or collection of computers or servers, or a cloud of computers or servers. Typically, a cluster or collection of servers can be used when the demand on computer system 100 exceeds the reasonable capability of a single server or computer. In certain embodiments, computer system 100 may comprise a portable computer, such as a laptop computer. In certain other embodiments, computer system 100 may comprise a mobile device, such as a smartphone. In certain additional embodiments, computer system 100 may comprise an embedded system.

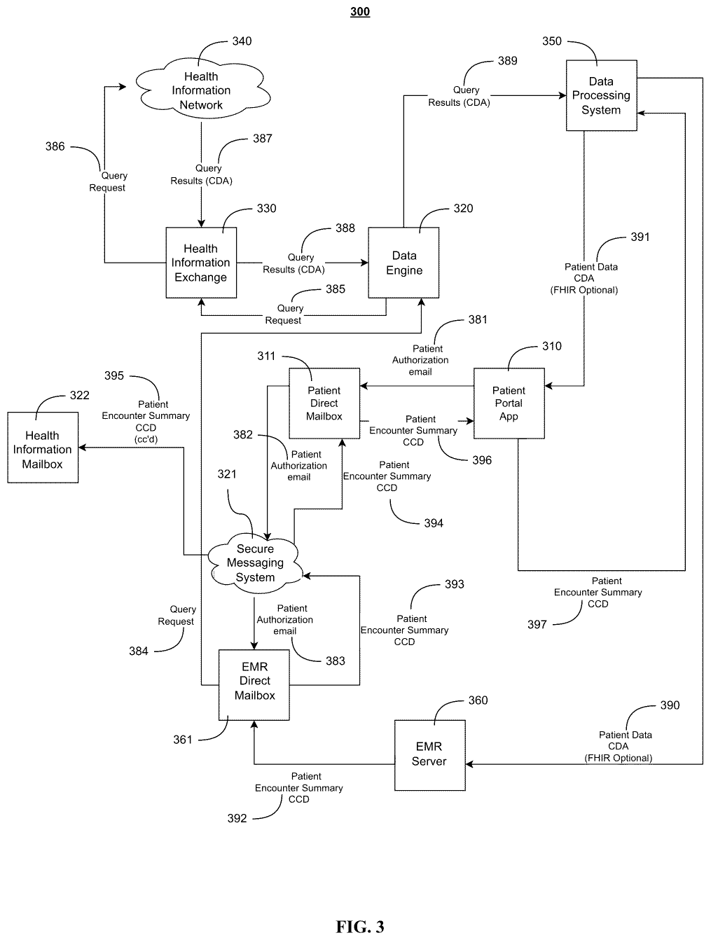

Turning ahead in the drawings, FIG. 3 illustrates a block diagram of a system 300 that can be employed for providing a patient healthcare utility and a data flow of messages between the components of system 300, according to an embodiment. System 300 is merely exemplary, and embodiments of the system are not limited to the embodiments presented herein. The system can be employed in many different embodiments or examples not specifically depicted or described herein. In some embodiments, certain elements, modules, or systems of system 300 can perform various procedures, processes, and/or activities. In other embodiments, the procedures, processes, and/or activities can be performed by other suitable elements, modules, or systems of system 300. The data flow of the messages (381-397) of FIG. 3 is merely exemplary. In some embodiments, the messages can be processed in the order presented. In other embodiments, the messages can be processed in any suitable order. In still other embodiments, one or more of the messages can be combined or skipped. In these or other embodiments, one or more of the messages can be sent as one or more computing instructions configured to run on one or more processors and configured to be stored on one or more non-transitory computer-readable media. Such non-transitory computer-readable media can be part of a computer system such as one or more of the components of system 300. The processor(s) can be similar or identical to the processor(s) described above with respect to computer system 100 (FIG. 1).

In some embodiments, such as shown in FIG. 3, system 300 can include a patient portal application (“app”) 310, a patient direct mailbox 311, a data engine 320, a secure messaging system 321, a health information mailbox 322, a health information exchange 330, a health information network 340, a data processing system 350, an electronic medical records (EMR) server 360, an EMR direct mailbox 361, and/or other suitable systems. Generally, therefore, system 300 can be implemented with hardware and/or software, as described herein. In some embodiments, part or all of the hardware and/or software can be conventional, while in these or other embodiments, part or all of the hardware and/or software can be customized (e.g., optimized) for implementing part or all of the functionality of system 300 described herein. The components of FIG. 3 can each be, or be implemented on, a computer system, such as computer system 100 (FIG. 1), as described above, and can each be, or be implemented on, a single computer, a single server, or a cluster or collection of computers or servers, or a cloud of computers or servers. In another embodiment, a single computer system can host some or all of the components of system 300.

In some embodiments, the components of system 300 can be in data communication with each other through one or more networks (not shown). For example, a user, such as a patient, a family member of a patient, or a healthcare provider, can use user devices, which can host patient portal app 310. The user device can be part of system 300 or external to system 300. The network can be the Internet or another suitable network. In many embodiments, patient portal app 310 can be an application, such as a mobile application or desktop application, or a web-based application. In many embodiments, data engine 320 can host one or more websites and/or mobile application servers that can be accessed by patient portal app 310. For example, data engine 320 can host a website, or provide a server that interfaces with patient portal app 310 on the user device, which can provide a portal for users to register, login, request EHRs, access EHRs, send EHRs, message healthcare providers, and/or other suitable activities, as described below in further detail. In various embodiments, the operator and/or administrator of data engine 320 can manage data engine 320, the processor(s) of data engine 320, and/or the memory storage unit(s) of data engine 320 using the input device(s) and/or display device(s) of data engine 320.

In certain embodiments, the user devices (e.g., the devices running patient portal app 310) can be desktop computers, laptop computers, mobile devices, and/or other endpoint devices used by one or more users. A mobile device can refer to a portable electronic device (e.g., an electronic device easily conveyable by hand by a person of average size) with the capability to present audio and/or visual data (e.g., text, images, videos, music, etc.). For example, a mobile device can include at least one of a digital media player, a cellular telephone (e.g., a smartphone), a personal digital assistant, a handheld digital computer device (e.g., a tablet personal computer device), a laptop computer device (e.g., a notebook computer device, a netbook computer device), a wearable user computer device, or another portable computer device with the capability to present audio and/or visual data (e.g., images, videos, music, etc.). Thus, in many examples, a mobile device can include a volume and/or weight sufficiently small as to permit the mobile device to be easily conveyable by hand. For examples, in some embodiments, a mobile device can occupy a volume of less than or equal to approximately 1790 cubic centimeters, 2434 cubic centimeters, 2876 cubic centimeters, 4056 cubic centimeters, and/or 5752 cubic centimeters. Further, in these embodiments, a mobile device can weigh less than or equal to 15.6 Newtons, 17.8 Newtons, 22.3 Newtons, 31.2 Newtons, and/or 44.5 Newtons.

Exemplary mobile devices can include (i) an iPod®, iPhone®, iTouch®, iPad®, MacBook® or similar product by Apple Inc. of Cupertino, California, United States of America, and/or (i1) a Galaxy™ or similar product by the Samsung Group of Samsung Town, Seoul, South Korea. Further, in the same or different embodiments, a mobile device can include an electronic device configured to implement one or more of (i) the iPhone® operating system by Apple Inc. of Cupertino, California, United States of America, and/or (ii) the Android™ operating system developed by the Open Handset Alliance.

In many embodiments, the components of system 300 can each include one or more input devices (e.g., one or more keyboards, one or more keypads, one or more pointing devices such as a computer mouse or computer mice, one or more touchscreen displays, a microphone, etc.), and/or can each comprise one or more display devices (e.g., one or more monitors, one or more touch screen displays, projectors, etc.). In these or other embodiments, one or more of the input device(s) can be similar or identical to keyboard 104 (FIG. 1) and/or a mouse 110 (FIG. 1). Further, one or more of the display device(s) can be similar or identical to monitor 106 (FIG. 1) and/or screen 108 (FIG. 1). The input device(s) and the display device(s) can be coupled to the components of system 300 in a wired manner and/or a wireless manner, and the coupling can be direct and/or indirect, as well as locally and/or remotely. As an example of an indirect manner (which may or may not also be a remote manner), a keyboard-video-mouse (KVM) switch can be used to couple the input device(s) and the display device(s) to the processor(s) and/or the memory storage unit(s). In some embodiments, the KVM switch also can be part of the components of system 300. In a similar manner, the processors and/or the non-transitory computer-readable media can be local and/or remote to each other.

Meanwhile, in many embodiments, the components of system 300 also can be configured to communicate with one or more databases. The one or more databases can be stored on one or more memory storage units (e.g., non-transitory computer readable media), which can be similar or identical to the one or more memory storage units (e.g., non-transitory computer readable media) described above with respect to computer system 100 (FIG. 1). Also, in some embodiments, for any particular database of the one or more databases, that particular database can be stored on a single memory storage unit or the contents of that particular database can be spread across multiple ones of the memory storage units storing the one or more databases, depending on the size of the particular database and/or the storage capacity of the memory storage units. The one or more databases can each include a structured (e.g., indexed) collection of data and can be managed by any suitable database management systems configured to define, create, query, organize, update, and manage database(s). Exemplary database management systems can include MySQL (Structured Query Language) Database, PostgreSQL Database, Microsoft SQL Server Database, Oracle Database, SAP (Systems, Applications, & Products) Database, and IBM DB2 Database.

Meanwhile, the components of system 300 and/or the one or more databases can be implemented using any suitable manner of wired and/or wireless communication. Accordingly, system 300 can include any software and/or hardware components configured to implement the wired and/or wireless communication. Further, the wired and/or wireless communication can be implemented using any one or any combination of wired and/or wireless communication network topologies (e.g., ring, line, tree, bus, mesh, star, daisy chain, hybrid, etc.) and/or protocols (e.g., personal area network (PAN) protocol(s), local area network (LAN) protocol(s), wide area network (WAN) protocol(s), cellular network protocol(s), powerline network protocol(s), etc.). Exemplary PAN protocol(s) can include Bluetooth, Zigbee, Wireless Universal Serial Bus (USB), Z-Wave, etc.; exemplary LAN and/or WAN protocol(s) can include Institute of Electrical and Electronic Engineers (IEEE) 802.3 (also known as Ethernet), IEEE 802.11 (also known as WiFi), etc.; and exemplary wireless cellular network protocol(s) can include Global System for Mobile Communications (GSM), General Packet Radio Service (GPRS), Code Division Multiple Access (CDMA), Evolution-Data Optimized (EV-DO), Enhanced Data Rates for GSM Evolution (EDGE), Universal Mobile Telecommunications System (UMTS), Digital Enhanced Cordless Telecommunications (DECT), Digital AMPS (IS-136/Time Division Multiple Access (TDMA)), Integrated Digital Enhanced Network (iDEN), Evolved High-Speed Packet Access (HSPA+), Long-Term Evolution (LTE), WiMAX, etc. The specific communication software and/or hardware implemented can depend on the network topologies and/or protocols implemented, and vice versa. In many embodiments, exemplary communication hardware can include wired communication hardware including, for example, one or more data buses, such as, for example, universal serial bus(es), one or more networking cables, such as, for example, coaxial cable(s), optical fiber cable(s), and/or twisted pair cable(s), any other suitable data cable, etc. Further exemplary communication hardware can include wireless communication hardware including, for example, one or more radio transceivers, one or more infrared transceivers, etc. Additional exemplary communication hardware can include one or more networking components (e.g., modulator-demodulator components, gateway components, etc.).

In a number of embodiments, when a new user (e.g., patient) begins to use patient portal app 310, patient portal app 310 can guide the user through a registration and self-authentication process. For example, the user can setup a username and password, and can upload a picture of a driver's license of the user and/or a current picture of the user, using patient portal app 310. Once the user is authenticated, patient portal app 310 can present terms and conditions to allow the user to agree to those terms and conditions.

Once the user has completed the initial registration process, patient portal app 310 can initiate a records request on behalf of the patient to request EHRs of the patient. In some embodiments, a patient direct mailbox 311 can be provided by secure messaging system 321 for access by the patient, such as through patient portal app 310. In a number of embodiments, patient direct mailbox 311 can be associated with a user. In some embodiments, patient portal app 310 can be used by the patient to send a message 381 from patient portal app 310 to patient direct mailbox 311. In some embodiments, message 381 can be a patient authorization email, which can request EHRs for the patient. In some embodiments, patient direct mailbox 311 can send a message 382 to secure message system 321, and/or secure message system 321 can send a message 383 to EMR direct mailbox 361. Message 382 and/or 383 can be similar or identical to message 381. In some embodiments, EMR direct mailbox can be provided by secure message system 321 for access by EMR server.

In a number of embodiments, EMR direct mailbox can sent a message 384 to data engine 320. In many embodiments, message 384 can be a query request for EHRs of the patient. In some embodiments, data engine 320 can send a message 385 to health information exchange 330, based on the records request initiated by the user. In a number of embodiments, data engine 320 act as a utility activated and used by the user, which can be activated for the user irrespective of the race, age, health conditions, etc., of the user. In many embodiments, data engine 320 and/or health information exchange 330 can include a healthcare data routing/interface engine, such as Mirth by NextGen Healthcare or another suitable routing/interface engine, to facilitate messaging of health-care data across various different platforms. Mirth can be included as an interoperability plug-in on data engine 320 and/or health information exchange 330. In several embodiments, health information exchange 330 can be an HIE (Health Information Exchange), such as C3HIE, HASA (Healthcare Access San Antonio), or another suitable HIE, which can make healthcare data available to healthcare providers. HIEs can allow healthcare providers, such as doctors, nurses, pharmacists, etc., to securely communicate and share EHRs.

In many embodiments, the messages can be exchanged using Direct Secure messaging, such as using DirectTrust addresses or another secure key provided by secure messaging system 321. In many embodiments, each patient can be setup with a Direct Secure address, which can provide a unique identifier for the patient. In some embodiments, an HISP (Health Information Service Provider), such as DataMotion or another suitable HISP, can be used to provide a certificate, in which the token is the address. The address can be used for the patient to communicate with a healthcare provider of the patient and/or to provide identification of the patient, such that the patient becomes a node on the HISP. The HISP can provide the secure messaging infrastructure. In many embodiments, data engine 320 can provision a new address for a new patient using the HISP when the patient is registered.

In many embodiments, health information exchange 330 can communicate with other HIEs through a health information network 340. In many embodiments, health information network 340 can be eHealth Exchange, or another suitable health information network to access third-party HIEs (Common Well, etc.). For example, health information exchange 330 can send one or more messages 386 to health information network 340. In some embodiments, message 386 can be a query request for EHRs for the patient. To access an HIE per HIPPA (Health Insurance Portability and Accountability Act) regulations, a request can come from a healthcare provider, a health system, or an HIE, which can query a HIE for PTO (payment, treatment, and/or operation). When a patient requests EHRs for the patient, the HIE can be obligated under the 21st Century Cures Act (Cures Act) regulations to return the EHRs within 36 hours. The patient also can request information about which entities have had access to the EHRs of the patient. In this way, a patient can readily request the EHRs for the patients across multiple HIEs. The EHRs collected from the HIEs (e.g., 330 and any third-party HIEs accessed through health information network 340) can be returned health information exchange 330, such as in one or more messages 387. In many embodiments, messages 387 can be query results, which can be EHRs in a CDA (Clinical Document Architecture), FHIR (Fast Healthcare Interoperability Resource), and/or other document standard. The CDA documents can be XML documents, while the FHIR documents can be web-based documents. CDA and FHIR can each be formatted according to its own data structure standard. In many embodiments, the EHR can be received in the format requested.

Additionally, health information exchange 330 can communicate with one or more payers (not shown), such as health insurance companies, to request healthcare payment records, such as explanation of benefits (EOB) information, and the payers can be under the same obligation under the Cures Act to provide the healthcare payment records within 36 hours. The records can be returned from HIEs accessed through health information network 340, through health information exchange 330 to data engine 320, such as in one or more messages 388. Messages 388 can be similar or identical to message 387.

Once data engine 320 has received one or more EHRs, the EHRs can be sent in one or more messages 389 to data processing system 350. Messages 389 can be similar or identical to messages 387 and/or 388. In many embodiments, data processing system 350 can perform data normalization, data duplication, correcting syntactical errors, and/or other suitable data processing, to transform the EHR in a format suitable for exporting the EHR to a provider and/or the patient. For example, in some cases, an EHR can be requested as a payload of particular fields, and the EHR can include the data in the payload in one of the data formats (e.g., CDA, FHIR). Data processing system 350 can transform the payload of the EHR to a format suitable for export to the provider and/or patient (e.g., CDA, FHIR, PDF (Portable Document Format), or another suitable data format, etc.). In some embodiments, data processing system 350 can be or include Diameter Health, which can provide NCQA (National Committee for Quality Assurance) certification for the exported EHRs, or another suitable data processing system. Data processing system 350 can tag the data for analytics, based on the codes in the data, which can allow the EHRs output to be analyzed more readily.

After performing the data processing, data processing system 350 can send the EHRs to EMR server 360 in one or more messages 390 and/or to patient portal app 310 in one or more messages 391. Patient portal app 310 can allow the user (e.g., patient) to request to view or send the EHRs to a healthcare provider. The patient can own the EHRs, which can be stored in patient records database, which can be provided by data engine 320 and/or accessed by patient portal app 310. In many embodiments, the EHRs can be stored in the patient records database by data engine 320 on behalf of the patient.

In a number of embodiments, EMR server 360 can be Flexcare EMR or another suitable EMR server. In some embodiments, the EHRs for the patient can be sent from EMR server 360 to EMR direct mailbox 361, such as in one or more messages 392, which can be similar or identical to message 390. In a number of embodiments, EMR direct mailbox 361 can send one or more messages 393 to secure messaging system 321. Messages 393 can be similar or identical to messages 390 and/or 392. In a number of embodiments, secure messaging system can sent the EHRs for the patient to patient direct mailbox 311, such as in one or more messages 394, and/or to a health information mailbox 322 in one or more messages 395. For example, health information mailbox 322 can be a mailbox provided by secure messaging system 321 that is accessible by health information exchange 330. In many embodiments, patient direct mailbox 311 can make the EHRs available to the patient in patient portal app 310, such a by sending one or more messages 396 from patient direct mailbox 311 to patient portal app 310.

In many embodiments, data engine 320 can provide a cloud-based healthcare utility. In a number of embodiments, patient portal app 310 and data engine 320 can be secured by encryption, and in some embodiments, can be secured by military-grade grid and/or lattice encryption, such as provided by Code-X, to provide security against hacking and/or identity theft of the EHRs and communications. In many embodiments, Code-X can use biometrics for authentication of the user. Patient portal app 310 and data engine 320 can empower users and families to take ownership of their personal EHRs, readily communicate with their care team, insurance companies, and/or family members. A “patient-centric” model can be used to enable patients to provide access to their provider and others they trust of their EHRs and/or complete electronic medical records. Access to complete medical records can help eliminate gaps in their care, drive down costs of care, reduce over-payment, and/or improve patient outcomes.

In several embodiments, patient portal app 310 and data engine 320 can rely on the patient's HIPPA rights to acquire consolidated medical records, which can provide patients with direct access to HIEs and allow patients to take ownership of their medical records. Patient portal app 310 and data engine 320 also can provide the ability to share actionable medical records, manage medical records for the entire family, and provide “at-a-glance” key medical records for providers (e.g., physicians). Patient portal app 310 and data engine 320 can be HIPPA-compliant, HITRUST certified, and/or NCQA certified. The patient portal app 310 and data engine 320 can be scalable to large populations. Patients using the patient portal app 310 and data engine 320 can be assigned a Direct Address, which is a truly unique identifier that allows the patient to communicate directly with providers through the same network that providers use to communicate with each other.

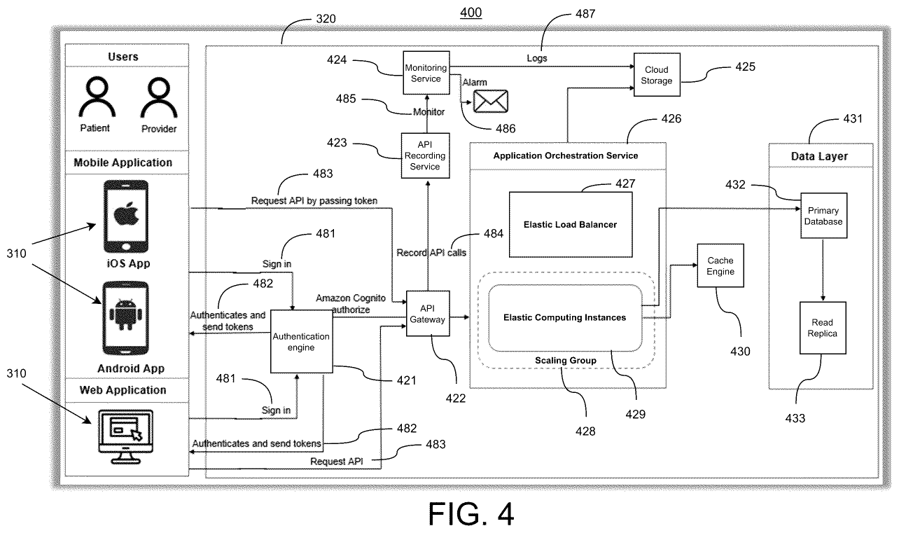

Turning ahead in the drawings, FIG. 4 illustrates a block diagram of a system 400 that can be employed for providing a patient healthcare utility and a data flow of activities involving the components of system 400, according to an embodiment. System 400 illustrates an implementation that can be used for patient portal app 310 and data engine 320, but other suitable implementations are contemplated in other embodiments. The data flow involving activities (481-488) is merely exemplary. In some embodiments, the activities can be performed in the order presented. In other embodiments, the activities can be performed in any suitable order. In still other embodiments, one or more of the activities can be combined or skipped. In these or other embodiments, one or more of the activities can be sent as one or more computing instructions configured to run on one or more processors and configured to be stored on one or more non-transitory computer-readable media. Such non-transitory computer-readable media can be part of a computer system such as one or more of the components of system 300. The processor(s) can be similar or identical to the processor(s) described above with respect to computer system 100 (FIG. 1).

In many embodiments, such as shown in FIG. 4, users (such as a patient or a provider) can access an application, such as a mobile application implemented as an IOS app running on an iPhone/iPad or implemented as an Android app running on an Android mobile device, or a web application accessed on a web browser running on a computer. The application can be similar or identical to patient portal app 310. The mobile applications can be developed using Xamarin or another suitable mobile application development platform. The application can used by the user to access the backend systems implemented in data engine 320. For example, data engine 320 can be implemented in Amazon Web Services or another suitable cloud-based environment. In many embodiments, data engine 320 can include an authentication engine 421, an API gateway 422, an API recording service 423, a monitoring service 424, cloud storage 425, an application orchestration service 426 (which can include an elastic load balancer 427, a scaling group 428 of clastic computing instances 429), a cache engine 430, a data layer 431 (which can include a primary database 432 and/or a read replica 433), and/or other suitable systems.

In many embodiments, such as shown in FIG. 4, the data flow can include one or more activities 481 in which the user signs in and user credentials are sent from patient portal app 310 to authentication engine 421 of data engine 320. For example, authentication engine 421 can be implemented by Amazon Cognito or another suitable service, which can validate and authenticate by returning access tokens (e.g., JWT (JSON (JavaScript Object Notation) Web Token)) in one or more activities 482. After successful authentication, patient portal app 310 can request an API (application programming interface) resource by passing the token in its authorization header in one or more activities 483 of requesting APIs. API requests from patient portal app 310 can be directed to API gateway 422, which can act as a single-entry point. API gateway 422 can be implemented by Amazon API Gateway, or another suitable service. Next, API gateway 422 can authorize the request with the help of authentication engine 421 and pass it to application orchestration service 426, which can process the request and/or serve the response back to the user. Application orchestration service 426 can be a logic layer for computing implemented by AWS Beanstalk, EC2, or another suitable service, and in some embodiments, there clastic load balancer 427 can be used to balance computing across scaling group 428 of clastic computing instances 429. The API related data can be recorded in an API recording service 423 (e.g., Amazon Cloud Trail or another suitable recording service), such as an activity 484. Monitoring service 424 (e.g., Amazon Cloud Watch or another suitable service) can perform one or more activities 485 of monitoring. Activity 486 or sending alarms/alerts, if necessary, and/or activity 487 of logging can be performed. In many embodiments, the logs can be saved in cloud storage 425. A cache engine 430 (e.g., ElastiCache for Redis or another suitable service) can be used for in-memory caching storage. Data layer 431 can store the data, such as in primary database 432 (e.g., Amazon RDS SQL server instance or another suitable database) and/or a backup archive, e.g., read replica 433.

The architecture shown in FIG. 4 can provide a number of benefits, while implementing authentication, authorization, security, scalability, high availability and/or performance. For example, the benefits can include identity management. The mobile users (also web users) can sign into the application via authentication engine 421. It is a user directory which can also be used for user onboarding (sign up) and takes care of authentication and authorization. One of the advantages of using the authentication engine pool is to allow users to sign-in with their social media (e.g., Facebook, Google or other social) accounts. This can also be integrated with other enterprise identity providers like a SAML provider. This service can scale up to millions of users which is advantageous because the users of the mobile application could be anyone in the U.S. who wants to maintain their health information.

The benefits also can include scalability. API gateway 422 can scale-out and scale-in dynamically without the need for manual intervention or configuration related to scalability parameters. It scales according to load seamlessly when integrated with lambda based on their own internal rules when there is a spike in demand. Data layer 431 can be scaled out by creating read only replicas (e.g., 433) of the primary instance (e.g., 432) which would be used for both read and write operations. This would also considerably reduce the load of the primary instance.

The benefits additionally can include security. Data can be protected at-rest by tokenization. The metrics can be collected in real time, and can be logged, with notifications if any action needs to be taken when there is a security threat. Encryption is enforced in transit by limiting what is allowed to secure protocols (HTTPS). This can be achieved by configuring a security group to allow HTTPS protocol in API gateway 422, data layer 431, and/or other services.

The benefits further can include an audit trail: A managed audit trail can be provided by API recording service 423, and the APIs which were requested through the API gateway can be automatically logged. This service can record and/or detect a wide range of actions including account activity, including actions taken through management consoles, software development kits, command line tools, and/or other services.

The benefits additionally can include monitoring. Monitoring service 424 can make it easy to monitor and analyze API usage. API gateway 422 can be monitored for a lot of metrics and the cloud watch logs can be saved in cloud storage 425. Also, as read replicas of the primary instance (e.g., 432) of data layer 431 are used, monitoring service 424 also can help to monitor the replication state and has necessary info about the replication lag between the primary database and the read replica instance.

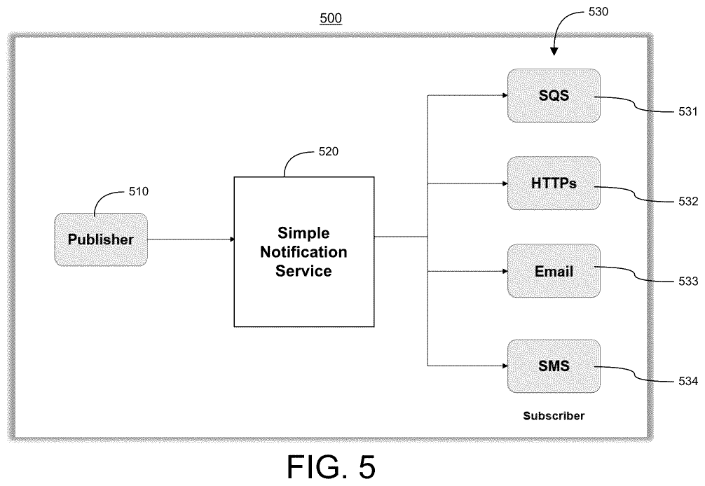

The benefits also can include mobile push notifications. For example, a SNS (simple notification service), such as shown in FIG. 5 and described below, can enable the logic tier to send real-time cross-device push notifications.

The benefits further can include disaster recovery. Can be achieved effectively by having automated backups of the database and configuring read replicas helps as well, because they are available in case of the failure of the primary database. Cross region support is available for read replica. Hence in case of any regional failure, the read replica in the different region can act as a standby database and can used as the primary instance thus ensuring business continuity.

Turning ahead in the drawings, FIG. 5 illustrates a flow chart 500 of a publish-subscribe messaging model using an SNS 520. SNS 520 can be web service that coordinates and manages the delivery or sending of messages to subscribing endpoints or clients and makes it easy to set up, operate, and send a notification from the cloud. It can use a publish-subscribe (pub-sub) messaging paradigm between a publisher 510 and one or more subscribers 530, with notifications being delivered to the client using a push mechanism that eliminates the need to periodically check or poll for new information and updates. SNS 520 also can send the messages to devices by sending push notifications to Apple, Google, Fire OS and Windows devices. A publisher 510 can be the entity that triggers the sending of a message (e.g., a monitoring service 424 alarm, any application running in application orchestration service 426, and/or cloud storage 425 events). Publishers 510 also are known as producers that produce and send the message to SNS 520 which is a logical access point. Subscriber 530 can be an endpoint to which a message is sent. Messages can be simultaneously pushed to subscriber 530. Subscribers 530 can include SQS queues 531, HTTP web servers 532, email addresses 533, SMS (short messaging service) 534, and/or other suitable functions to receive the messages or notifications, e.g., AWS Lambda. Subscribers subscribe to the topic to receive the message.

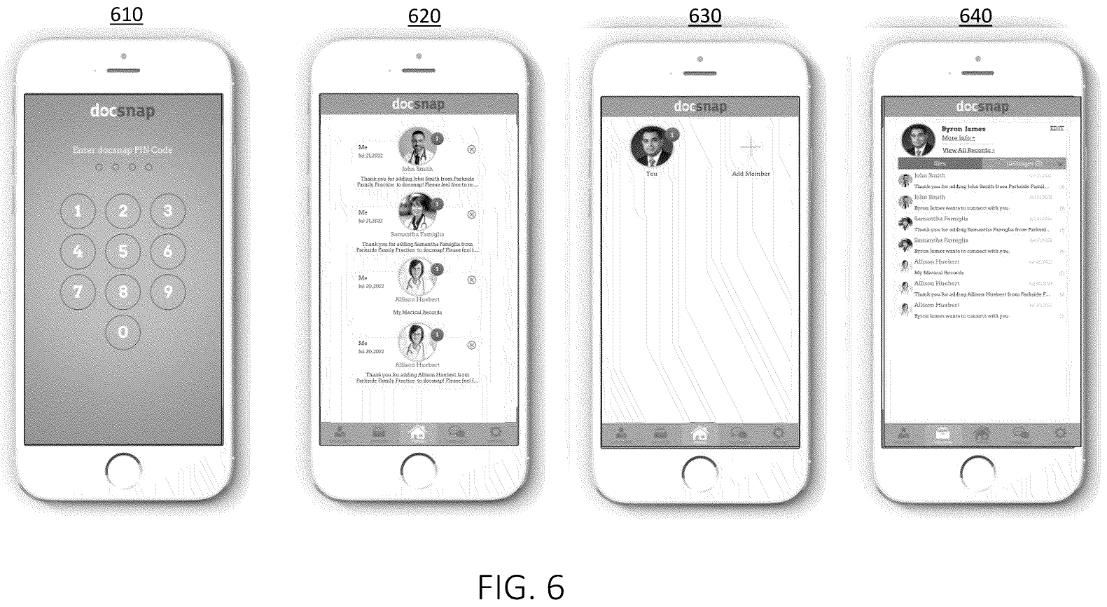

Turning ahead in the drawings, FIGS. 6-13 illustrate exemplary display screens on a mobile application, such as patient portal app 310 (FIG. 3) used by a patient. As shown in FIG. 6, a login screen 610 can ask the user (e.g., a patient) for a PIN (personal identification number) code. Once logged in, a display screen 620 can show notifications, such as providers that have been added, or new medical records that are available. A user account screen 630 can show the account of the user, and allow the user to add other account, such as for family members. A files and messages screen 640 can display files and/or messages, such as new or recent files and/or messages.

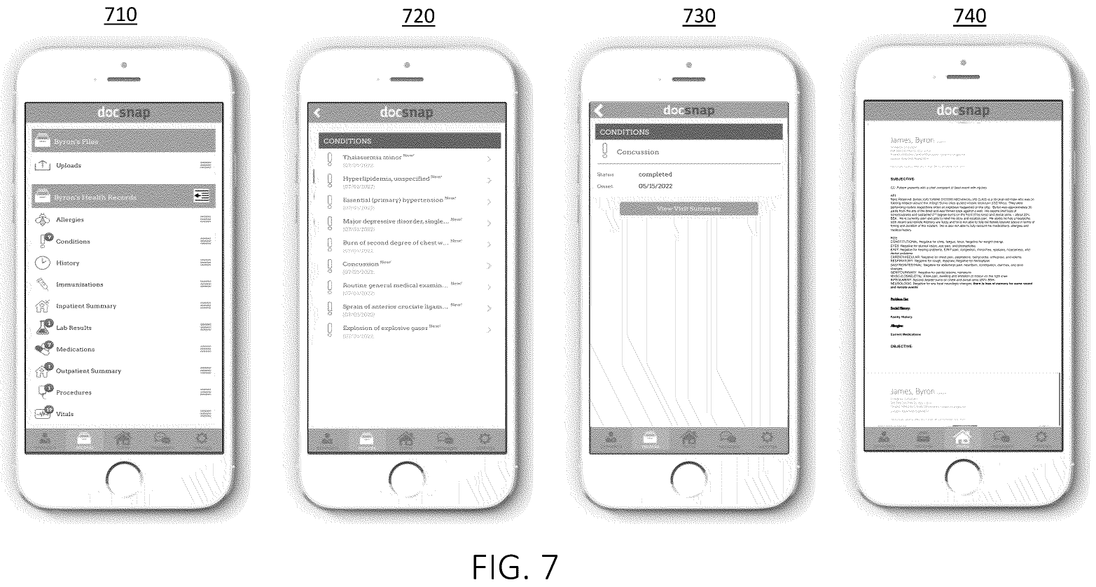

Turning ahead in the drawings, FIG. 7 shows a C-CDA (Consolidated CDA) screen 710, which can display healthcare records, or summaries thereof, for the user, which can be categorized according to various categories, such as allergies, conditions, history, immunizations, impatient summary, lab results, medications, outpatient summary, procedures, vitals, and/or other suitable categories. The user can select one of these categories to display further details, such as selecting conditions to display a conditions listing page 720, which can list medical conditions that apply to the user. The user can select one of these conditions to display one or more condition details pages, such as showing the status and onset date of the condition on a condition status page 730, and/or details of the condition on a condition details page 740.

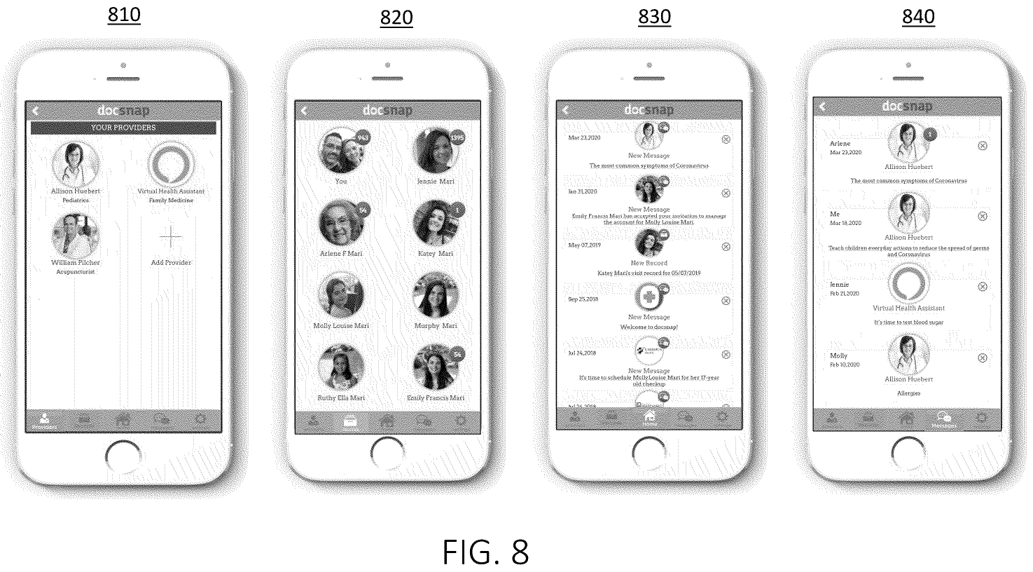

Turning ahead in the drawings, FIG. 8 shows a provider screen 810, which can show healthcare providers for a user, such as doctors, nurses, pharmacists, and/or other healthcare providers. The user can select one of these healthcare providers to contact the provider, request a virtual appointment, and/or perform other activities related to the provider. The mobile app can display a family members screen 820, which can allow users to view and/or add their family members and/or other loved ones to the app, such that all of the EHRs for a family can be accessed in the app by a single user without needing to login separately for different users. When there are multiple family members, all of the providers for the family members can be shown on the provider screen. A home screen 830 can show recent messages or notifications. A messages screen 840 can show notifications or messages from providers.

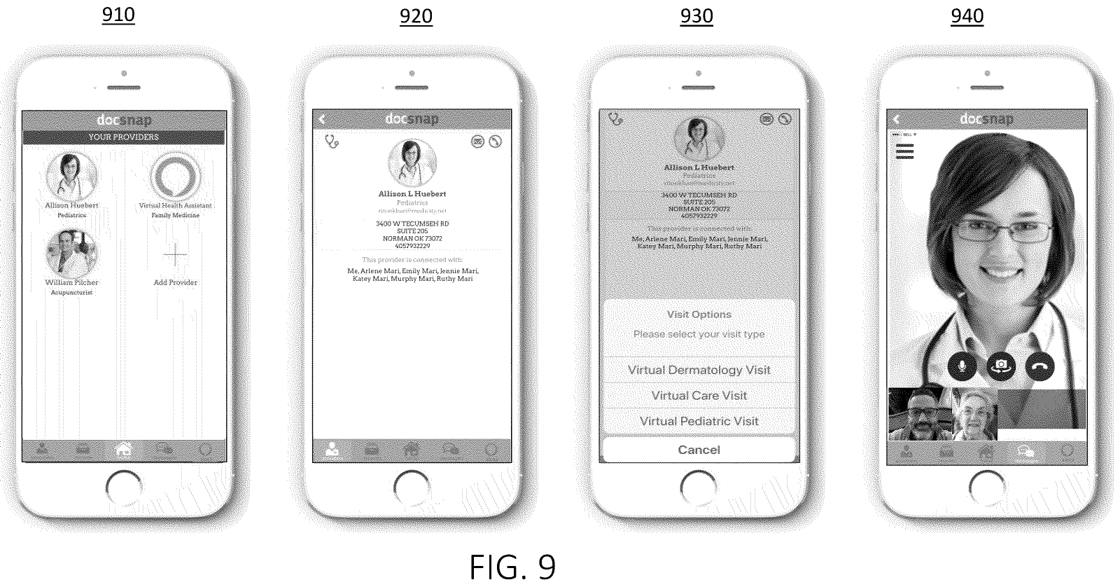

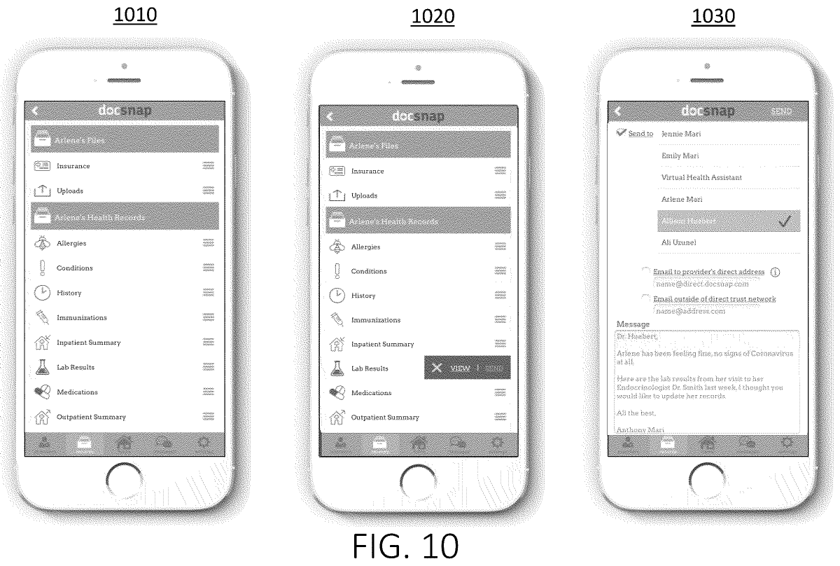

Turning ahead in the drawings, FIG. 9 shows a provider screen 910, on which a user can select a provider to show details about that provider on a provider details screen 920. Provider details screen 920 can show information for a particular provider, such as email address, office address, and/or which individuals in the family associated with that provider. Provider details screen 920 also can include buttons to send a direct message to the provider through the mobile application, setup a visit with the provider, call the provider through the mobile application, and/or disconnect the provider from the listing of providers for the user in the mobile application. For example, selecting a stethoscope logo can result in the application displaying visit options 930, such as a virtual dermatology visit and/or other types of visits. The application also can be used to facilitate a secure virtual visit 940, such as a video visit, for the user and/or a family member with the provider. Among others, the family members can include individuals who have not signed up for a user account, such as children and/or aging parents. The user can connect the account of the user with such family members to receive notifications and/or messages for such family members. When the family member also has an account of their own, both the family member the user can receive messages and/or notifications for the family member when the accounts are connected. The user also can view medical records for the family member, such as by selecting the family member on family members screen 820 in FIG. 8, then the app displaying a C-CDA screen (e.g., 710 (FIG. 7), or 1010 (FIG. 10, described below)) for or that family member, which can be used to display further details, such as displaying condition details pages (e.g., 730, 740 (FIG. 7)).

Turning ahead in the drawings, FIG. 10 shows a C-CDA screen 1010 that can include an option button for each category of medical records, and/or for the complete medical records for a user or family member, which, when selected can display options to view and/or send the medical records. For example, if the user selects the send button for lab results, as shown in an action screen 1020, the user can then select providers that are linked to the user or family member, and/or can enter email addresses of other providers, as shown in a message screen 1030. Additionally, the user can enter a message in message screen 1030 to accompany the records. The records and/or message can then be sent to the selected providers. Users can send their EHRs to their providers with the same authority that another provider on the network can, and the EHRs can be in a format that is acceptable, usable, auditable, and/or actionable by the provider.

In some embodiments, the mobile application can be integrated with a voice assistant, such as Alexa, to provide a virtual health assistant. Navigation through the application can be achieved through voice commands. For example, the user can audibly request opening the application, after which the application can notify the user that there is a new message and audibly ask if the message should be read. If the user asks for the message to be read, the application can audibly read the message, and then audibly prompt for other directions and/or audibly provide other information, such as scheduling an appointment with a provider and/or the user asking and receiving information about when a most-recent eye exam occurred. As another example, the user can audibly request and/or receive messages about insurance, such as insurance deductible payments, status toward total out-of-pocket spending, etc. As yet another example, the user can audibly request prescription refills, transfer prescriptions to another pharmacy provider (e.g., Pill Pack), and/or receive additional information about prescription medications.

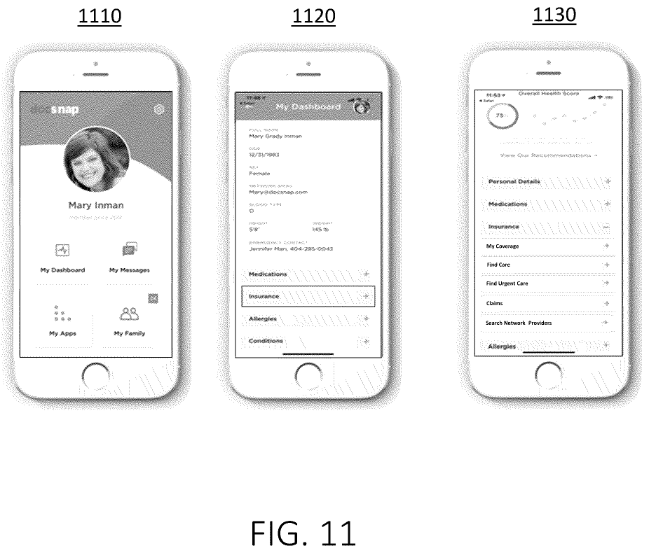

Turning ahead in the drawings, FIG. 11 shows a home screen 1110 for a user, which can be used to navigate to a dashboard screen 1120. On dashboard screen 1120, the user can select insurance, which be used to open up additional options related to insurance, as shown in an insurance options screen 1130, such as insurance coverage information, finding care accepted by the insurer, finding urgent care accepted by the insurer, viewing claims, searching for in-network providers, and/or other suitable options.

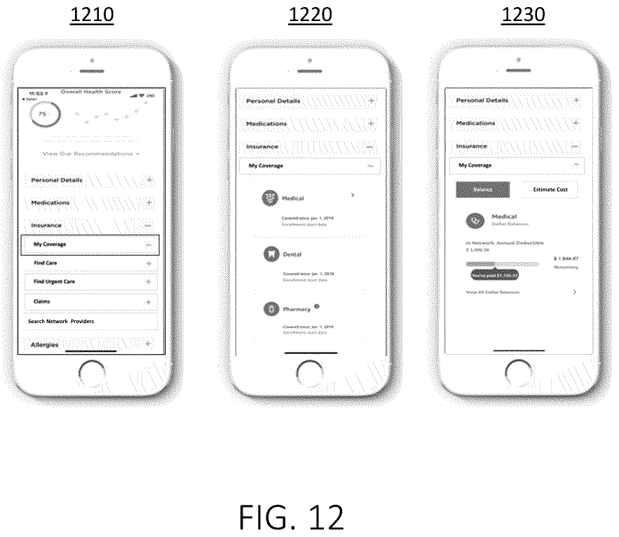

Turning ahead in the drawings, FIG. 12 shows a user selecting an option to display insurance coverage information in an insurance options screen 1210 (which can be similar to insurance options screen 1130 (FIG. 11)), which, when selected, can provide display a coverage screen 1220, which can present options to select the type of insurer, such as medical, dental, and/or pharmacy. For example, if the user selects medical, a medical coverage screen 1230 can show the medical insurer, the balances for the in-network annual deductible and/or other balances.

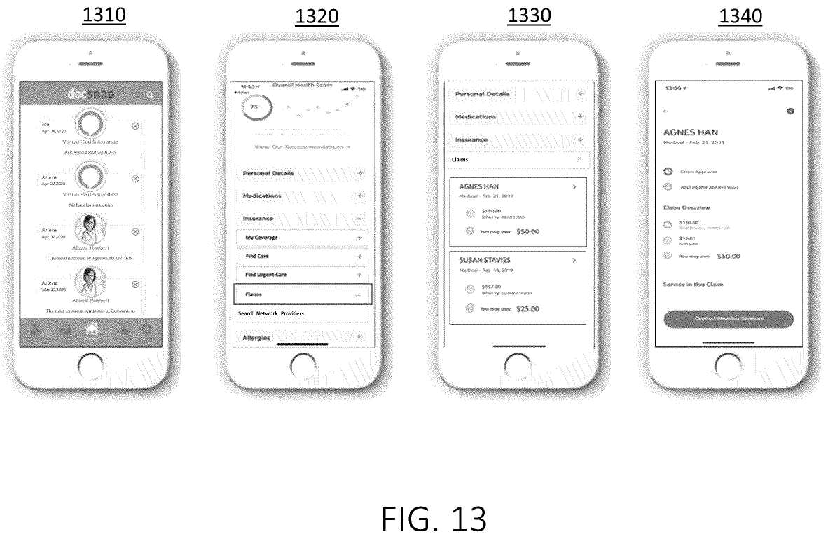

Turning ahead in the drawings, FIG. 13 shows a messages screen 1310 for messages or contacting a virtual health assistant. FIG. 12 also shows a user selecting the option to view claims in a menu screen 1320, which, when selected, can provide a claims screen 1330 that lists claims along with a claims summary, such as provider name, date of service, claim amount, and/or amount owed. Selecting an individual claim can result in the application providing additional information about the claim on a claim details screen 1340, such as if the claim was approved or denied, the covered individual, how much was billed by the provider, how much the plan paid, how much is owed, details of service in the claim, and/or other suitable information. In some embodiments, the application can provide a button to allow the user to contact member services for the claim.

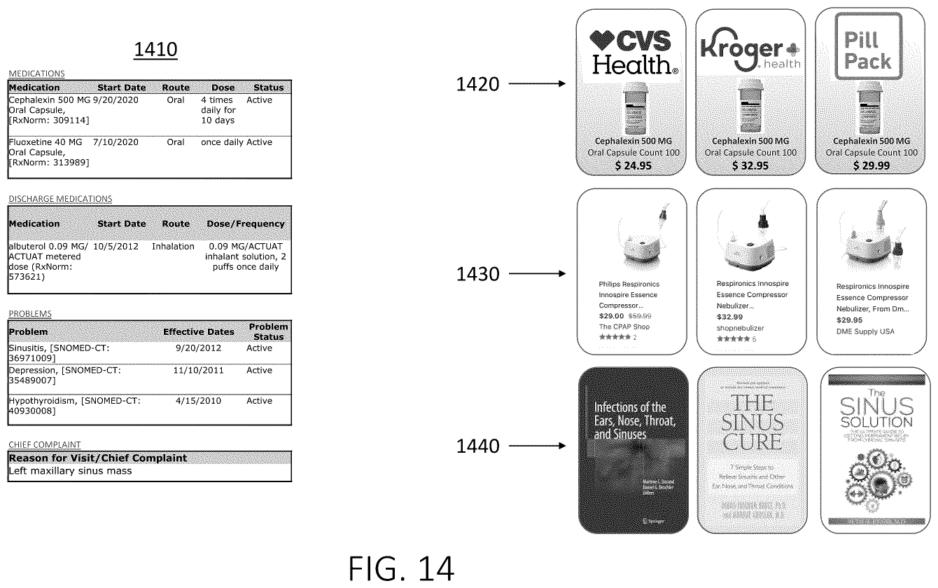

Turning ahead in the drawings, FIG. 14 illustrates how clinical data can be actionable in the application. FIG. 14 displays a CDA 1410 in human-readable format, which includes a list of prescribed medications, discharge medications, problems, and a chief complaint. The application and/or the backend can analyze the clinical data to drive transactions in a competitive marketplace, which can drive down the cost of healthcare and provide consumers with choices. For example, the application and/or the backend can determine that there is a prescription for cephalexin, and can search for and display prescription options 1420 to the user for purchasing cephalexin at various different retailers. As another example, the application and/or the backend can determine that there is a prescription for albuterol, and can search for and display administration options 1430 to the user for purchasing nebulizers to use to administer the albuterol. As yet another example, the application and/or the backend can determine that a problem is sinusitis, and the application and/or the backend can use the actionable clinical data to drive prevention and wellness plans along with educational materials, such as by displaying plans and/or materials 1440.

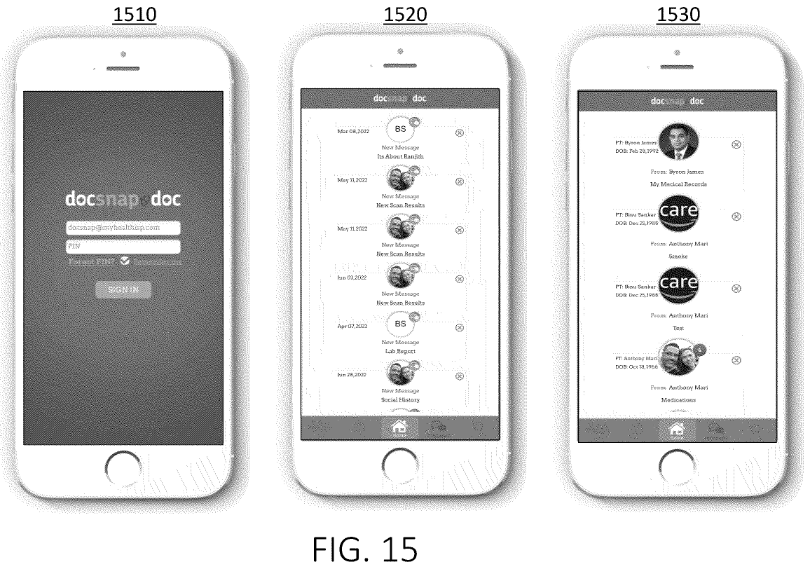

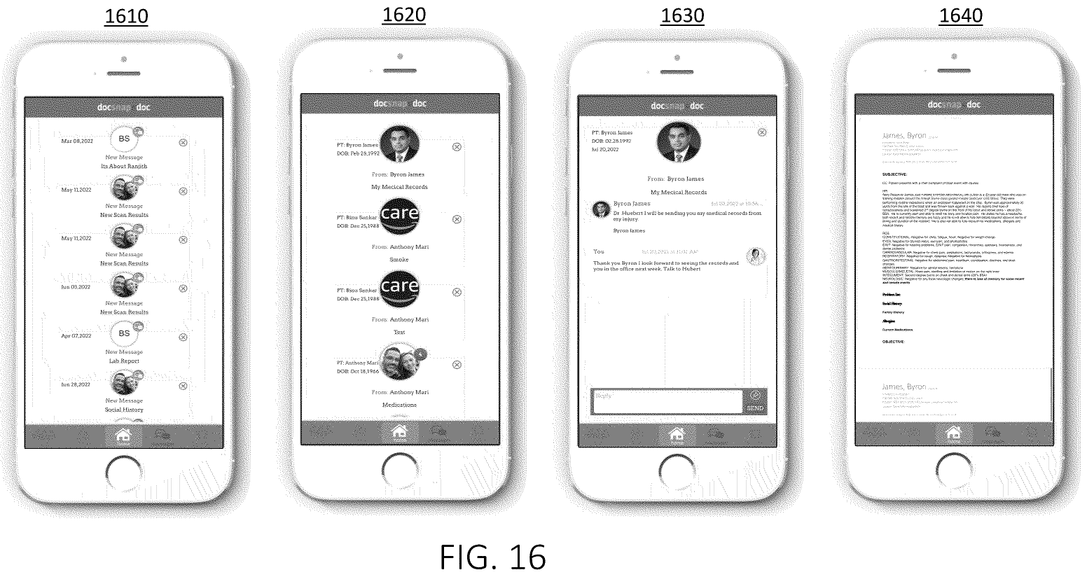

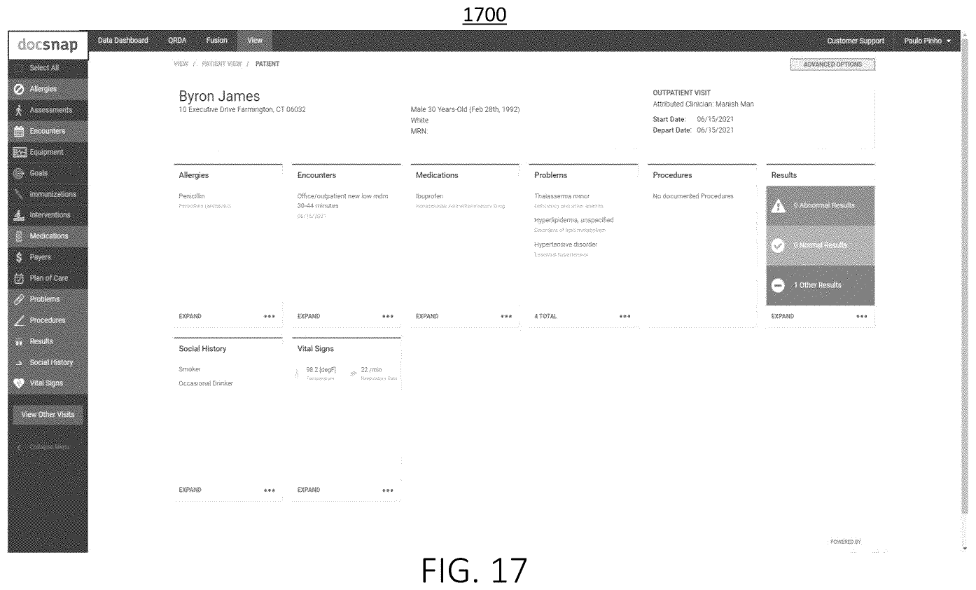

Turning ahead in the drawings, FIGS. 15-16 illustrate exemplary display screens on a mobile application used by a provider, and FIGS. 17-19 illustrate exemplary display screens on a web application used by a provider. As shown in FIG. 15, a login screen 1510 can ask the user (e.g., the provider) for a username and PIN. Once logged in, a messages screen 1520 can show messages from patients, new lab results, etc. Patients can share their EHRs, individually, by category, or consolidated with any provider, such as shown in a sent records screen 1530, and the EHRs can readily be imported into any provider EHR system in a standard healthcare data format. For providers that are connected to the application, the provider also can access the EHRs through the application.

Turning ahead in the drawings, FIG. 16 shows how, when a provider selects a message on one of message listing screens 1610 or 1620, the application can display the message from the patient to the provider on a message screen 1630 and can allow the provider to send a message in response to the patient, such as shown in a send message screen 1640. For example, the patient can send medical records related to an injury to a provider in advance of an appointment, the provider can access the medical records on a medical records screen, and/or the provider can respond to the patient. The application and the backend thus can provide direct communications between patients and providers, to facilitate personal, secure, and timely communication.

Turning ahead in the drawings, FIG. 17 shows a webpage view 1700 that displays a summary view of patient longitudinal medical records, which can expedite patient assessment and accurate decision-making at the point-of-care. For example, summaries of patient information, allergies, encounters, medications, problems, procedures, results, social history, and vital signs can be displayed. The webpage view can be an interactive dashboard, such that selecting one of these categories can allow the provider to view additional information about that category.

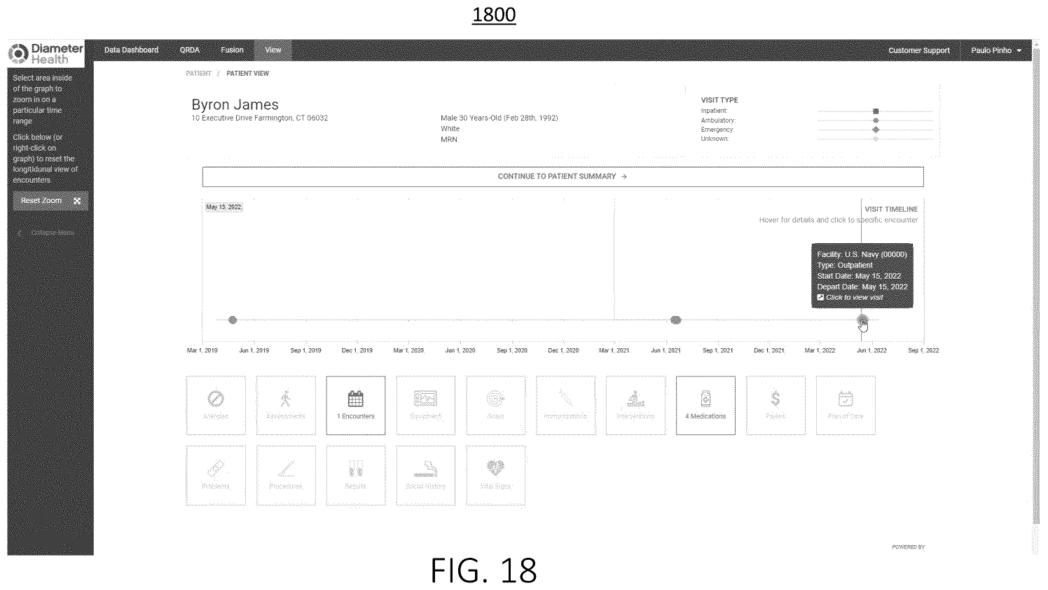

Turning ahead in the drawings, FIG. 18 shows a webpage view 1800 that displays a timeline view, which can enable the provider to readily see the patient's medical history and access specific encounters within that history. For example, the provider can hover over and/or click on a point in the timeline to view additional information about an encounter.

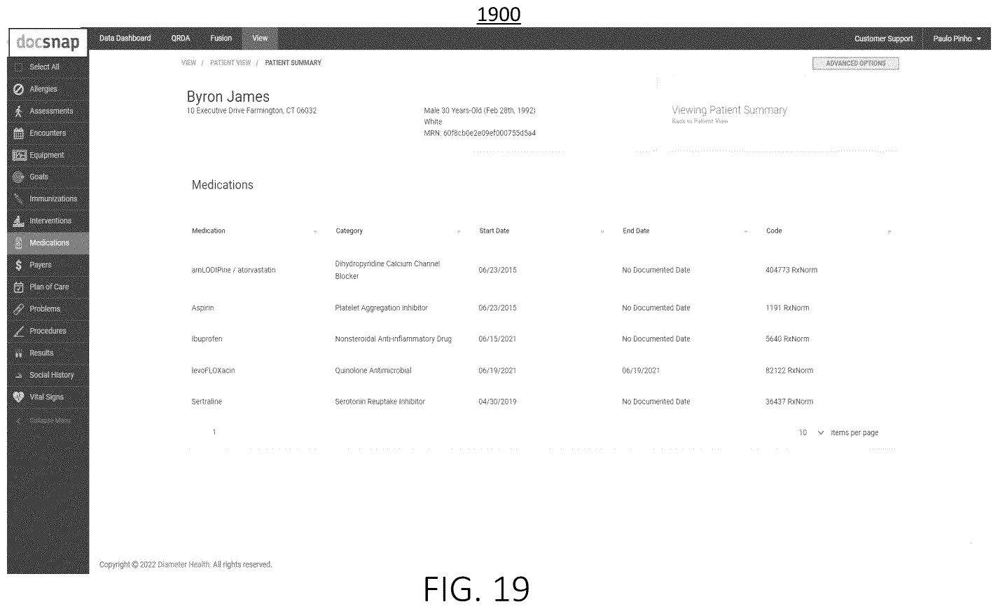

Turning ahead in the drawings, FIG. 19 shows a webpage view 1900 that displays medications for a patient. The provider can select a category of medical records to view such information, such as a list of current and/or past medications. The medical records can be owned by the patient, yet can be actionable and/or auditable, so as to be capable of being used at the point-of-care by the provider to provide treatment.

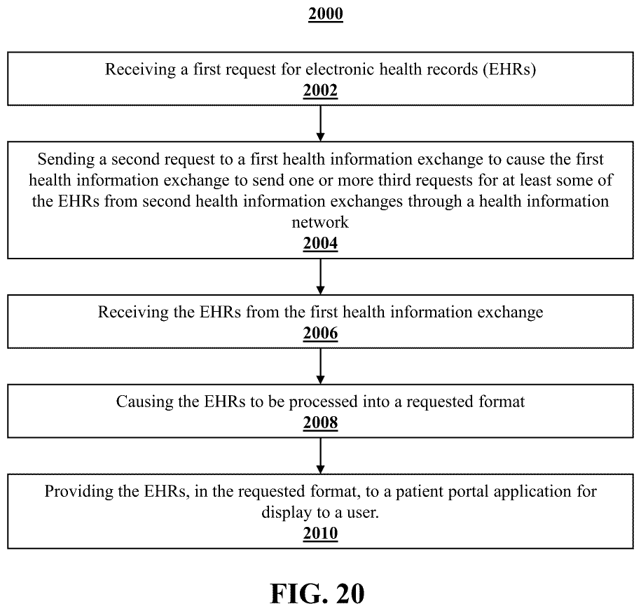

Turning ahead in the drawings, FIG. 20 illustrates a flow chart for a method 2000 of retrieving and providing EHRs through a patient healthcare utility, according to an embodiment. Method 2000 is merely exemplary, and embodiments of the process are not limited to the embodiments presented herein. The method can be employed in many different embodiments or examples not specifically depicted or described herein. In some embodiments, certain steps, activities, or procedures of method 2000 can be performed by various systems, modules, or components. In other embodiments, the steps, activities, or procedures can be performed by other suitable systems, modules, or components. Method 2000 can be performed by a system similar or identical to data engine 320 (FIG. 3).

In some embodiments, such as shown in FIG. 20, method 2000 can include an activity 2002 of receiving a first request for EHRs. This request may be initiated by a user, such as a patient, through a patient portal application or other suitable interface.

Method 2000 also can include an activity 2004 of sending a second request to a first health information exchange. The first health information exchange can be similar or identical to health information exchange 330 (FIG. 3). The second request can be based on the first request. Activity 2004 can cause the first health information exchange to send one or more third requests to other health information exchanges through a health information network for at least some of the EHRs. The health information network can be similar or identical to health information network 340 (FIG. 3).

In several embodiments, method 2000 additionally can include an activity 2006 of receiving the EHRs from the first health information exchange. The EHRs received can be in various formats, such as CDA, FHIR, or other suitable document standards.

In a number of embodiments, method 2000 further can include an activity 2008 of causing the EHRs to be processed into a requested format. Activity 2008 can involve data normalization, data deduplication, correcting syntactical errors, and/or other suitable data processing to transform the EHR into a format suitable for exporting to a provider and/or the patient. For example, data system 320 (FIG. 3) can make a call to data processing system 350 (FIG. 3) to process the EHRs into the requested format. In many embodiments, the requested format can be provided by data engine 320 (FIG. 3) and/or patient portal app 310 (FIG. 3).

Method 2000 additionally can include an activity 2010 of providing the EHRs, in the requested format, to a patient portal application for display to a user. The patient portal application can be similar or identical to patient portal app 310 (FIG. 3).

Method 2000 demonstrates a sequence of steps involved in retrieving EHRs from multiple sources, processing them, and presenting them to the user through a patient portal application. It enables patients to access, manage, and share their health information while ensuring compliance with relevant healthcare regulations and standards.

In many embodiments, the techniques described herein can beneficially provide several technical advantages. As healthcare systems scale and become more complex, the challenges of EHR management increase. Numerous solutions exist for EHR storage and retrieval, emphasizing data security and interoperability, yet often overlooking the aspect of efficient data access and presentation to end-users. In developing healthcare applications with complex, relational schemas featuring multiple data layers from various sources, there is significant benefit to using the techniques described herein to manage EHRs in a manner that allows for rapid retrieval, access, scalability, case-of-use, and performance.

The techniques described herein can seamlessly integrate with existing health information exchange systems. They can transform disparate EHR data into a unified, accessible format, enabling efficient retrieval and presentation of related health information, addressing the challenge of consolidating patient data from multiple sources. In many embodiments, these techniques can offer standardized operations for requesting, processing, and presenting EHRs, applicable uniformly across various healthcare data models. By utilizing a centralized request and processing system, these techniques can update associated data across multiple health information exchanges, achieving automatic consolidation of patient records. In real-time healthcare applications with multiple users and providers, these techniques can handle data flow seamlessly, automatically determining the appropriate data sources and facilitating automatic updates to patient records and user interfaces.

The techniques can enhance the performance, reliability, and scalability of EHR management in large-scale healthcare applications. The generalizability of these techniques allows them to be applicable across diverse healthcare scenarios, from individual patient care to population health management. By providing a streamlined process for EHR retrieval, processing, and presentation, these techniques can significantly improve the efficiency of healthcare delivery, enhance patient engagement, and support better clinical decision-making.

The techniques described herein can beneficially address several technical problems. For example, the techniques described here can address the technical problem of data fragmentation, in which patient records are stored across multiple systems, providers, and formats. This technique addresses this problem by providing a unified method to request and aggregate EHRs from various health information exchanges through a centralized process. By sending requests to multiple sources and consolidating the responses, it creates a comprehensive view of a patient's health information.

Moreover, the techniques described herein can beneficially address the technical problem of interoperability. Different healthcare systems often use incompatible data formats and structures, making it difficult to exchange and integrate information. The described process tackles this issue by incorporating a data processing step that transforms the received EHRs into a requested format. This standardization enables seamless data exchange and presentation across different systems and applications.

Further, the techniques described herein can beneficially address the technical problem of real-time data access. In healthcare, timely access to patient information is advantageous. The technical solution provides a streamlined process for requesting, retrieving, and presenting EHRs, enabling real-time access to up-to-date patient information. This solution can particularly benefit clinical settings where quick decision-making is advantageous.

Additionally, the techniques described herein can beneficially address the technical problem of scalability. As healthcare systems grow and the volume of patient data increases, scalability becomes a significant technical challenge. The described technique addresses this issue by using a modular approach, separating the processes of data request, retrieval, processing, and presentation. This architecture allows for easy scaling of individual components as needed.

Further, the techniques described herein can beneficially address the technical problem of user interface complexity. Presenting complex medical data in a user-friendly manner is a technical challenge. By processing the EHRs into a requested format and providing them to a patient portal application, this solution enables the development of more intuitive and responsive user interfaces for both patients and healthcare providers.

Moreover, the techniques described herein can beneficially address the technical problem of data security and privacy. The use of health information exchanges and standardized data processing can implement secure data transfer and handling protocols, addressing the technical specifications for patient data privacy and security.

These techniques can provide a comprehensive technical solution that addresses multifaceted challenges of EHR management in modern healthcare systems. It can offer a scalable, interoperable, and efficient approach to handling patient data, thereby improving the overall performance and effectiveness of healthcare information technology.

Although the methods described above are with reference to the illustrated flowchart, it will be appreciated that many other ways of performing the activities associated with the method can be used. For example, the order of some operations may be changed, and some of the operations described may be optional.

In addition, the methods and system described herein can be at least partially embodied in the form of computer-implemented processes and apparatus for practicing those processes. The disclosed methods may also be at least partially embodied in the form of tangible, non-transitory machine-readable storage media encoded with computer program code. For example, the steps of the methods can be embodied in hardware, in executable instructions executed by a processor (e.g., software), or a combination of the two. The media may include, for example, RAMs, ROMs, DVD-ROMs, BD-ROMs, hard disk drives, flash memories, or any other non-transitory machine-readable storage medium. When the computer program code is loaded into and executed by a computer, the computer becomes an apparatus for practicing the method. The methods may also be at least partially embodied in the form of a computer into which computer program code is loaded or executed, such that, the computer becomes a special purpose computer for practicing the methods. When implemented on a general-purpose processor, the computer program code segments configure the processor to create specific logic circuits. The methods may alternatively be at least partially embodied in application specific integrated circuits for performing the methods.

The foregoing is provided for purposes of illustrating, explaining, and describing embodiments of these disclosures. Modifications and adaptations to these embodiments will be apparent to those skilled in the art and may be made without departing from the scope or spirit of these disclosures.

Although systems and methods for patient healthcare utility have been described with reference to specific embodiments, it will be understood by those skilled in the art that various changes may be made without departing from the spirit or scope of the invention. Accordingly, the disclosure of embodiments of the invention is intended to be illustrative of the scope of the invention and is not intended to be limiting. It is intended that the scope of the invention shall be limited only to the extent required by the appended claims. For example, to one of ordinary skill in the art, it will be readily apparent that any element of FIGS. 1-20 may be modified, and that the foregoing discussion of certain of these embodiments does not necessarily represent a complete description of all possible embodiments. For example, one or more of the procedures, processes, or activities of FIGS. 3-5 and 20 may include different procedures, processes, and/or activities and be performed by many different modules, in many different orders. As another example, the systems within FIGS. 3-4 can be interchanged or otherwise modified.

Replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

What is claimed is:

1. A computer-implemented method comprising:
receiving a first request for electronic health records (EHRs);
sending a second request to a first health information exchange to cause the first health information exchange to send one or more third requests for at least some of the EHRs from second health information exchanges through a health information network;
receiving the EHRs from the first health information exchange;
causing the EHRs to be processed into a requested format; and
providing the EHRs, in the requested format, to a patient portal application for display to a user.

2. The computer-implemented method of claim 1, further comprising authenticating the user prior to receiving the first request for the EHRs.

3. The computer-implemented method of claim 1, wherein the requested format is one of Clinical Document Architecture (CDA), Fast Healthcare Interoperability Resources (FHIR), or Portable Document Format (PDF).

4. The computer-implemented method of claim 1, further comprising storing the EHRs in a patient records database.

5. The computer-implemented method of claim 4, wherein the patient records database is accessible by the patient portal application.

6. The computer-implemented method of claim 1, further comprising receiving healthcare payment records comprising explanation of benefits (EOB) information from one or more payers.

7. The computer-implemented method of claim 1, wherein the third requests are requested by the first health information exchange.

8. A system comprising one or more processors and one or more non-transitory computer-readable media storing computing instructions that, when executed on the one or more processors, cause the one or more processors to perform operations comprising:
receiving a first request for electronic health records (EHRs);
sending a second request to a first health information exchange to cause the first health information exchange to send one or more third requests for at least some of the EHRs from second health information exchanges through a health information network;
receiving the EHRs from the first health information exchange;
causing the EHRs to be processed into a requested format; and
providing the EHRs, in the requested format, to a patient portal application for display to a user.

9. The system of claim 8, wherein the operations further comprise authenticating the user prior to receiving the first request for the EHRs.

10. The system of claim 8, wherein the requested format is one of Clinical Document Architecture (CDA), Fast Healthcare Interoperability Resources (FHIR), or Portable Document Format (PDF).

11. The system of claim 8, wherein the operations further comprise storing the EHRs in a patient records database.

12. The system of claim 11, wherein the patient records database is accessible by the patient portal application.

13. The system of claim 8, wherein the operations further comprise receiving healthcare payment records comprising explanation of benefits (EOB) information from one or more payers.

14. The system of claim 8, wherein the third requests are requested by the first health information exchange.

15. One or more non-transitory computer-readable media storing computing instructions that, when executed on one or more processors, cause the one or more processors to perform operations comprising:
receiving a first request for electronic health records (EHRs);
sending a second request to a first health information exchange to cause the first health information exchange to send one or more third requests for at least some of the EHRs from second health information exchanges through a health information network;
receiving the EHRs from the first health information exchange;
causing the EHRs to be processed into a requested format; and
providing the EHRs, in the requested format, to a patient portal application for display to a user.

16. The one or more non-transitory computer-readable media of claim 15, wherein the requested format is one of Clinical Document Architecture (CDA), Fast Healthcare Interoperability Resources (FHIR), or Portable Document Format (PDF).

17. The one or more non-transitory computer-readable media of claim 15, wherein the operations further comprise storing the EHRs in a patient records database.

18. The one or more non-transitory computer-readable media of claim 17, wherein the patient records database is accessible by the patient portal application.

19. The one or more non-transitory computer-readable media of claim 15, wherein the operations further comprise receiving healthcare payment records comprising explanation of benefits (EOB) information from one or more payers.

20. The one or more non-transitory computer-readable media of claim 15, wherein the third requests are requested by the first health information exchange.
